(19) United States
(12) Patent Application Publication
(10) Pub. No.: US 2024/0228471 A1
(43) Pub. Date: Jul. 11, 2024

(54) MOF, MOF LINKERS AND MANUFACTURING METHOD THEREOF

(71) Applicant: UNIVERSITEIT ANTWERPEN, Antwerpen (BE)

(72) Inventors: Christophe VANDE VELDE, Antwerpen (BE); Rosa FUCCI, Antwerpen (BE); Leili ESRAFILI-DIZAJI, Antwerpen (BE)

(21) Appl. No.: 18/555,985

(22) PCT Filed: Apr. 22, 2022
(86) PCT No.: PCT/EP2022/060673

§ 371 Date: Oct. 18, 2023

(30) Foreign Application Priority Data
Apr. 23, 2021 (EP) 21170206.3

(51) Int. Cl. C07D 409/14; B01J 31/16

(52) U.S. Cl. CPC C07D 409/14; B01J 31/1691; B01J 2531/37

(57) ABSTRACT

The present invention relates to Metal Organic Frameworks (MOF), linkers for said MOFs and method of manufacturing thereof, wherein the MOF linker comprises a core and spacers having aryl moieties.

### FIELD OF THE INVENTION

The present invention relates to the field of Metal Organic Frameworks (MOF), linkers for said MOFs and method of manufacturing thereof. More specifically, the present invention pertains to MOF linkers comprising a core and spacers, each having aryl moieties which are connected to one another by means of direct C—C bonds.

### BACKGROUND TO THE INVENTION

New green regulations and policies are expected to get stricter and stricter in the incoming decades, requiring industries to adopt a more sustainable approach. Among several industries, the chemical industry's journey to sustainability is only in its early stages. As the world's population nears nine billion, and the strain on the planet's resources grows, in order to ensure that the future of better chemistry is a success, the development of innovative environmentally friendly catalysts is of crucial importance for the establishment of a new sustainable chemical industry. Catalysts can be divided in two classes, homogeneous catalysts and heterogeneous catalysts. The greatest advantage of heterogeneous catalysts is their ease of separation, while the disadvantages are often limited activity and selectivity, which are more to be found in homogeneous catalysts.

The heterogenization of homogeneous catalysts generates novel catalysts that would show the advantages of both the classes of chemical catalysts: high activity and selectivity and possibility to recover the catalyst. Among the classes of porous materials used for heterogeneous catalysis (zeolites, silicas, etc.), MOFs have a unique combination of characteristics that makes them the most suitable material to serve as a scaffold for the immobilization of metal catalysts and their use as personalizable nanoreactors for catalyzed reaction in pharmaceutical, agrochemical and fine chemical industry.

MOFs can be made to exhibit the perfect combination of properties (crystallinity, easy functionalization, large pore size, high surface area), and appear to be suitable scaffolds for use in the heterogenization of homogeneous catalysts. The current gap for the effective use of homogeneous catalysts in a heterogeneous way is the synthesis of novel and large-pore MOF structures that can immobilize a metal catalyst complex, complete with the activating ligand, and leave enough free space for reagent and product diffusion.

The use of organic linkers for the manufacturing of MOFs is preferred over other manufacturing methods as it allows the design of MOFs ab initio, having control over the final MOF to be obtained by synthesizing specific suitable linkers. The synthetic path for such molecular linkers is often based on a high number of synthetic steps, making difficult the use of the material for applications mainly because of the low overall yields and highly expensive products. Due to the need for customizable catalytic scaffold, the synthesis of the linkers shall be modular, so that different parts can be easily selected and used in order to generate libraries of molecules without time-consuming and expensive processes.

Kajiwara et al., 2017, discloses MOFs made from MOF linkers comprising an aryl core and spacers comprising aryl moieties, more specifically substituted thiophenes. Kajiwara et al., 2017, discloses MOF linkers synthesized via Suzuki-Miyaura coupling reactions between the corresponding tris(bromo(hetero)aryl)benzenes and the (hetero) arylboronic acids bearing carboxy substituents, see supplementary information. The disadvantage of the method of manufacturing of MOF linkers according to Kajiwara et al., 2017, is that expensive boron comprising reagents are necessary, which have to be synthesized, adding steps and complexity to the synthesis of MOF linkers.

In the industry there is the need for MOF linkers, and MOFs comprising said linkers, which are cheaper to produce, which require less manufacturing steps, and can be obtained via a modular approach. The present invention aims at overcoming drawbacks of the prior art.

### SUMMARY OF THE INVENTION

In a first aspect, the present invention relates to a method of manufacturing of a linker, comprising the steps of:
a) providing a molecular core X comprising n first aryl moieties having a group A attached thereto, wherein n≥2, and wherein the group A is provided to form a C—C bond after catalytic activation with a cross coupling metal catalyst,

The core X is fast and cheap to synthesize following a high yielding one step reaction with easy purification steps (e.g. recrystallization). Having a fixed core lays the basis to implement libraries of linkers that differ from each other in the spacers. This makes possible to build large libraries with less effort.

Therefore, the present step a) comprises providing a molecular core X which is substituted with aryl moieties which can undergo direct arylation (form a C—C bond after catalytic activation). Each n first aryl moieties therefore comprises at least a group A, so that in the whole molecular core at least n group A's are present. The present invention further provides the step:
b) providing at least n spacers S, each comprising at least a second aryl moiety having a group B attached thereto, and wherein the group B is provided to form a C—C bond after catalytic activation with the cross coupling metal catalyst, and wherein each of said n spacers S is substituted with a group C according to formula (D):

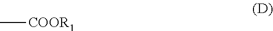

wherein
R₁ is selected from the list: H, an alkyl group, preferably methyl;

Therefore, the present step b) comprises providing at least an amount n of spacers S (thus at least equal to the amount of group A's and first aryl moieties in the molecular core X) each comprising a group B which is provided to form a C—C bond after catalytic activation with the same cross coupling metal catalyst as the one used to activate group A. In this way, both the group A and the group B can be catalytically activated with a cross coupling metal catalyst, to form a C—C bound between the core X and the spacers S, at the position of said groups. Further, the present invention comprises the step:
c) catalytically activating group A of the molecular core X and group B of the spacers S;

In accordance with the present step, group A of the molecular core X and group B are catalytically activated.

Further, the present invention comprises the step d) of:
d) Reacting via direct arylation in a solvent the molecular core X comprising n first aryl moieties having a group A with n second aryl moieties part of the spacers S, thereby forming at least a C—C bond at the position of said group A's and group B's, and wherein the solvent is an ether, preferably a cyclic ether.

Therefore, the present step d) comprises reacting the core X with the spacers S via direct arylation, with the formation of a direct C—C bond between a first aryl moiety and a second aryl moiety. In a direct arylation reaction, group A and B are removed and a direct C—C bond is formed. The solvent wherein the molecular core X and the spacers S are reacted is an ether, preferably a cyclic ether. An advantage of the present invention is that a cheap and fast synthesis of organic linkers can be achieved. Further, the present invention provides a method for achieving a modular synthetic approach.

In an embodiment according of the present invention, at step b) R₁ is an alkyl group, preferably methyl. An advantage of the present embodiment is that the methyl is a simple alkyl group and can be included and removed with a fast non-metal-catalysed high yielding reaction.

In an embodiment according to the present invention, at steps a) to d) n equals to 3 or 4.

In an embodiment according to the present invention, at steps a) to d) n equals to 3. An advantage of the present embodiment is that higher yields can be achieved if 3 substitutions are required to be performed on a molecule instead of 4, and hence purification is also easier.

In an embodiment according of the present invention, at step d) the solvent is tetrahydrofuran (THF), also known as oxolane.

In a further embodiment according of the present invention, at step b) the second aryl moiety is substituted with a group C of formula (D). Therefore, after the direct arylation reaction of step d) each of the n spacers S comprise a group C which is connected to the aryl group having a direct C—C bond with the first aryl moiety of the core X.

In an embodiment according of the present invention, at step b) at least one spacer S, preferably all spacers S, comprise an amino group. The amino group can be mono- or di substituted (—NHR or —NR₂) or unsubstituted (—NH₂).

In an embodiment according of the present invention, at step b) at least one spacer S, preferably all spacers S, comprise an amino group as defined herein and/or a group C of formula (D) attached to the second aryl moiety. In accordance with the present embodiment, the second aryl moiety of the spacer S, which is the moiety which is directly connected to the core X via direct arylation, comprises amino group and/or a group C of formula (D).

In a preferred embodiment according of the present invention, the second aryl moiety is a thiophene ring. An advantage of the present embodiment is that step d) is easier to perform.

In an embodiment according of the present invention, at steps a) to d), for each molecular core X, respectively spacer S, groups A and B are each independently selected from either H or Y, wherein Y is a direct arylation leaving group, such as a halide, such as Cl, Br, F, I, preferably Br, a triflate, a tosylate or a pseudohalide.

In a further embodiment according of the present invention, the second aryl moiety is a thiophene ring and B is H. Therefore, in accordance with the present embodiment, the thiophene ring is part of the spacer S and the group on the thiophene ring which is adapted to be removed during direct arylation is a hydrogen atom.

In an embodiment according of the present invention, at step d), the molar ratio of n first aryl moieties to spacers S is at least 1:1, preferably 1:2.

In a second aspect, the present invention pertains to a method of manufacturing of a MOF, the method comprising the steps a) to d) according to any one of the foregoing embodiments, and further comprising the steps of:
e) providing, if not already provided at step b), a free carboxylic group(s) (—COOH) from the group C according to formula (D) on any one, preferably all, of the spacers S;
f) coordinating a metal ion or cluster (M) with one or more of the free carboxylic groups provided at step e) or b), preferably all, of said spacers S, thereby forming the MOF.

In a third aspect, the present invention pertains to a linker obtained according to the method of any one the foregoing embodiments, comprising:
a molecular core X comprising n first aryl moieties, and
n spacers S, each comprising at least a second aryl moiety connected by means of direct C—C bond to any one of the first aryl moieties, and wherein each spacer S comprises a group C according to formula (D):

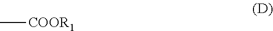

wherein
R₁ is selected from the list: H, an alkyl group, preferably methyl;
n≥2.

In an embodiment according to the present invention, in the linker R₁ is an alkyl group, in particular C₁₋₆alkyl, preferably methyl.

In an embodiment according to the present invention, in the linker n equals to 3 or 4.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1, also referred to as FIG. 1, illustrates N₂ sorption isotherms for La-16-1, La-16-2 and La-16-3.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. When describing the compounds of the invention, the terms used are to be construed in accordance with the following definitions, unless a context dictates otherwise.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

In a first aspect, the present invention relates to a method of manufacturing of a linker for MOFs, comprising the steps of:
a) providing a molecular core X comprising n first aryl moieties having a group A attached thereto, wherein n≥2, and wherein the group A is provided to form a C—C bond after catalytic activation with a cross coupling metal catalyst.

In accordance with the present invention, by means of the term “MOF” or “metal organic Framework”, reference is made to a class of polymeric crystalline materials comprising both organic and inorganic building blocks. More specifically, MOFs comprise a linker, which is an organic linker, and metal ion or cluster M which is coordinated by means of said linker. In some cases the MOF linkers in combination with some specific metal cores (e.g. tritopic linkers and La(III), tetratopic linkers and Zr clusters, etc.) can play the main role in establishing the topology of the MOFs.

In accordance with the present invention, by means of the term “linker” or “MOF linker”, reference is made to a molecular entity serving as a scaffold for the immobilization of metal ions or metal clusters M which can for example have a catalytic function. A linker is composed of a molecular core X and at least two spacers S. Linkers can be classified according to the number of spacers S extending from a common molecular core X, so that e.g. tritopic linkers are linkers comprising tritopic cores and 3 spacers S connected thereto, and tetratopic linkers are linkers comprising tetratopic cores and 4 spacers connected thereto.

In accordance with the present invention, by means of the term “molecular core X” or “core X”, reference is made to a molecular entity providing attaching point for spacers S departing therefrom. In general, the molecular core X gives the spatial configuration of the final linker. Molecular cores providing anchorage for 3 spacers S are called tritopic cores, whereas Molecular cores providing anchorage for 4 spacers S are called tetratopic cores. Examples of suitable molecular cores X are shown here below, see (I, II, IV, IX, X, XI), wherein A represents one or more groups attached to an aryl portion of said core X. An aryl moiety of the core X can comprise one or more A groups.

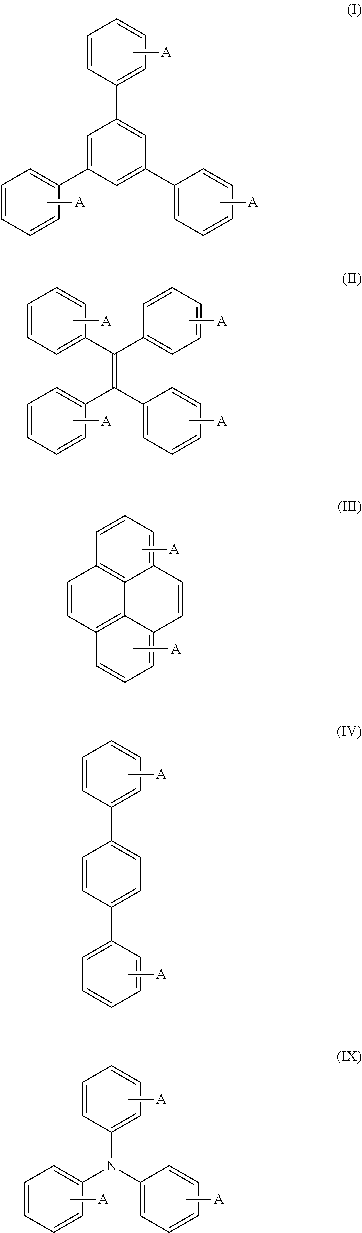

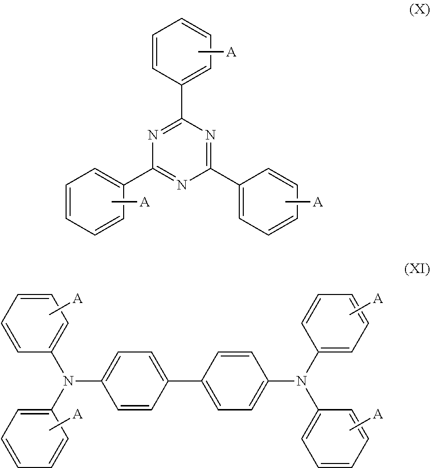

In accordance with an embodiment of the present invention, the molecular core X is selected from 1,1,2,2-Tetraphenylethylene, Pyrene, para-Terphenyl, Triphenylamine, 2,4,6-Triphenyl-1,3,5-triazine, N,N,N′,N′-Tetraphenylbenzidine and derivatives thereof.

In accordance with the present invention, by means of the term “aryl moiety”, such as a first aryl moiety and a second aryl moiety, reference is made to any functional group or substituent derived from an aromatic ring, or heteroaromatic ring (if heteroatoms are present within the ring). Examples of aryl moieties are thiophene, phenyl, furan, pyrrole, N-substituted pyrrole and selenophene.

In accordance with the present invention, by means of the term “alkyl” by itself or as part of another substituent refers to a fully saturated hydrocarbon of Formula CxH2x+1 wherein x is a number greater than or equal to 1. Generally, alkyl groups of this invention comprise from 1 to 20 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄ alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers, heptyl and its isomers, octyl and its isomers, nonyl and its isomers; decyl and its isomers. C₁₋₆ alkyl includes all linear, branched, or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3-, or 4-methylcyclopentyl, cyclopentylmethylene, and cyclohexyl.

In accordance with the present invention, by means of the term “group A”, reference is made to a chemical group which is present onto the aryl moiety of the core X. Group A can be positioned at various locations of the core, and it is preferably positioned with respect to other A groups attached to said core so that a symmetric core X is provided. Groups A in accordance to the present invention are provided to form a C—C bond after catalytic activation with a cross coupling metal catalyst. Depending on the reaction conditions and the nature of the core, different groups A can be used, such as H or Y, wherein Y is a direct arylation leaving group, such as a halide, such as Cl, Br, F, I, preferably Br, a triflate, a tosylate or a pseudohalide. For example, a possible molecular core X is 1,3,5-Tri(4-bromophenyl)benzene, a tritopic core wherein groups A are Br atoms.

In accordance with the present invention, by means of the term “cross coupling metal catalyst”, reference is made to a catalyst, such as a Palladium catalyst, which is capable of providing aryl-aryl bond formation via C—H activation.

Therefore, the present step a) comprises providing a molecular core X which is substituted with aryl moieties which can undergo direct arylation (form a C—C bond after catalytic activation). Each n first aryl moieties therefore comprises at least a group A, so that in the whole molecular core at least n group A are present. In accordance with the present invention not necessarily all aryl moieties making the molecular core X have to comprise a group A, nevertheless, in accordance with the present invention the core X has to comprise at least two aryl moieties and at least two groups A, in other words, n equals to at least 2. For n=3 tritopic linkers can be obtained, for n=4 tetratopic linkers can be obtained.

Further, the method of manufacturing according to the present invention comprises the step of
b) providing at least n spacers S, each comprising at least a second aryl moiety having a group B attached thereto, and wherein the group B is provided to form a C—C bond after catalytic activation with the cross coupling metal catalyst, and wherein each of said n spacers S is substituted with a group C according to formula (D):

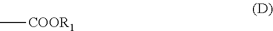

wherein
   R₁ is selected from the list: H, an alkyl group, preferably methyl.

In accordance with the present invention, by means of the term “spacer S”, reference is made to molecular entity extending from a molecular core, such as molecular core X, and comprises functional groups capable of interacting with a metal ion or cluster M. In general, the selection of a spacer S is directly correlated to the size of the MOF linker which in turn affects the pore size.

In accordance with the present invention, by means of the term “group B”, reference is made to a chemical group which is present onto the aryl moiety of the spacer S. In accordance with the present invention, the group B which is attached to the second aryl moiety and it is provided to form a C—C bond after catalytic activation with the same cross coupling metal catalyst as the one used to activate the group A, can be selected from H or Y, wherein Y is a direct arylation leaving group, such as a halide, such as Cl, Br, F, I, preferably Br, a triflate, a tosylate or a pseudohalide. For example, possible spacers S in accordance with the present invention can comprise thiophene and phenyl aromatic rings as second aryl moiety, wherein the spacer molecule comprises a C group and at least one group B attached to a second aryl moiety of the spacer molecule. The group B and the group C can be connected to the same aryl moiety, such as for (V), or a different aryl moiety, such as for (VI) and (VII). More specifically, the group C can be directly attached to the second aryl moiety, which is connected to the core X, or not. The group B is always attached to the second aryl moiety, but the group C can be attached to another part of the spacer molecule, including a further aryl moiety part of the spacer or a non aryl moiety.

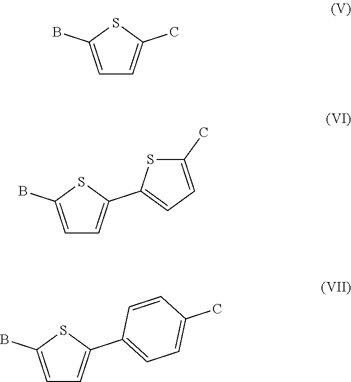

In accordance with the present invention, by means of the term “group C”, reference is made to a chemical functional group located onto a spacer S which is adapted to interact with a metal ion or cluster M.

In accordance with the present invention, by means of the term “ester moiety”, reference is made to a moiety comprising a carboxylate functionality (—(C═O)—O—R), or in equivalent notation —COOR.

Therefore, the present step b) comprises providing at least an amount n of spacers S (equal to the amount of group A and first aryl moieties) each comprising a group B which is provided to form a C—C bond after catalytic activation with the cross coupling metal catalyst. In this way, both the group A and the group B can be catalytically activated with a cross coupling metal catalyst.

Therefore, further, the method according to the present invention comprises the step:
c) catalytically activating group A of the molecular core X and group B of the spacers S

In order to catalytically activate group A and group B, various catalysts can be used, for example Pd(OAc)₂, Pd(PPh₃)₂Cl₂, Pd₂(dba)₃, preferably Pd(OAc)₂.

The cross coupling metal catalyst according to the present invention can be a metal catalyst of a metal selected from the list comprising, in order of decreasing preference: Palladium (Pd), Copper (Cu), Nickel (Ni), Iron (Fe), Ruthenium (Ru), Rhodium (Rh), Cobalt (Co).

According to a preferred embodiment of the present invention, the cross coupling metal catalyst is of metal being Palladium.

The present invention further comprises a step d), wherein at step d) the molecular core X and the n spacers S are reacted together via direct arylation. More specifically, step d) comprises
d) reacting via direct arylation in a solvent the molecular core X comprising n first aryl moieties with n second aryl moieties part of the spacers S, thereby forming at least a C—C bond at the position of said group A of the molecular core X and group B of the spacers S, and wherein the solvent is an ether, preferably a cyclic ether.

In accordance with the present invention, by means of the term “direct arylation”, reference is made to the synthetic organic reaction wherein an aryl-aryl bond is formed via C—H activation. This feature provides for several advantages. By means of a direct arylation reaction, the synthesis of MOF linkers is simpler. The synthesis can be achieved by coupling the C backbones of a first and a second aryl moiety through direct arylation. In conventional C—C coupling reactions both coupling partners are functionalized, e.g. in the well-known Suzuki coupling, one arene is substituted with —B(OR)₃ while the other one contains a halogen (I, Br). Approaches known in the prior art, such as ones including Suzuki coupling, would require extra synthetic steps compared to the method according to the present invention, as direct arylation is the coupling of e.g. aryl halides directly to a catalytically activated aryl C—H position. Therefore, the number of synthetic steps is reduced.

Therefore, the present step d) comprises reacting the core X with the spacers S via direct arylation, with the formation of a direct C—C bond between a first aryl moiety and a second aryl moiety. In a direct arylation reaction, group A and B are removed and a direct C—C bond is formed. For example in case the core X is a triphenyl core X with n=3 first aryl moieties, such as in (I), and the spacer S is a thiophene ring, such as in (V), in order to form a MOF linker according to the present invention, the triphenyl core X (I) is reacted with n=3 spacers S (V). The groups A and B, respectively attached to the core X (I) and at least a part of said spacer S (V) group A and B are removed during the direct arylation reaction and a direct C—C bond is formed between the molecular core X and the spacer S, see formula (VIII).

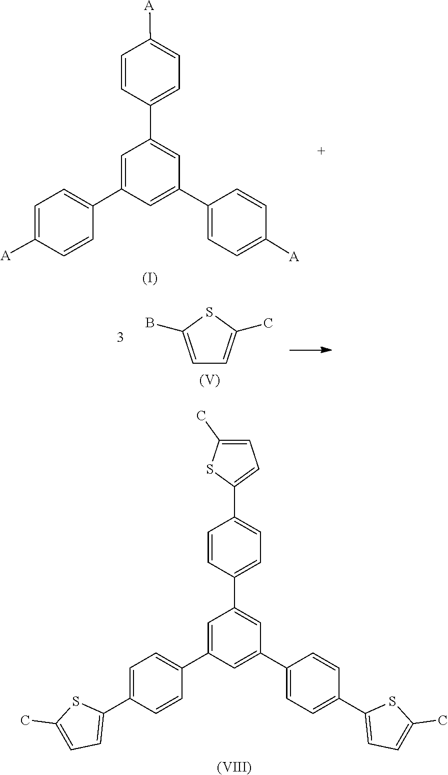

For the direct arylation reaction at step d), other than a cross coupling metal catalyst, such as Pd(OAc)₂ or Pd(PPh₃)₂Cl₂, a base, such as Cs₂CO₃ or K₂CO₃, a ligand to the metal catalyst Pcy-HBF₄, and a co-catalyst such as pivalic acid (PivOH) can be used. Metal catalyst PdCl(C3H5)(dppb) and KOAc as base. Metal catalyst Pd₂(dba)₃ P(o-MeOPh)₃ as ligand to the metal catalyst, a base such as Cs₂CO₃ or K₂CO₃ and PivOH as co-catalyst.

The present invention provides for several advantages, in particular, the method according to the present invention is that a cheap and fast synthesis of MOF linkers can be achieved. Further, the present invention provides a method for achieving a modular synthetic approach.

In particular, the synthesis of the MOF linkers according to the present invention allows to synthesize MOF linkers from two separate parts, a molecular core X and at least two spacers S which are preferably identical, but not necessarily. By means of this approach it becomes easier to generate molecular libraries for MOFs linkers which can be based on the selection of a reduced number of building blocks (2, a core X and a spacer S) compared to prior synthetic approaches. It has been found that the method according to the present invention is especially useful when a group C according to formula (D), —COOR₁ (D), attached to each spacer S, is present. It has been found that direct arylation is chemically tolerant to the presence of groups C onto a spacer S. The presence of a group C and a direct arylation reaction allows for less steps being needed to synthesize suitable MOF linkers and MOFs.

Further, in accordance with the present invention, a robust synthesis of MOF linkers is achieved, which is provided to function regardless of the composition of the chosen building blocks. This is achieved by the use of direct arylation of said core X with at least 2 spacers S.

In an embodiment according of the present invention, at step b) R₁ is an alkyl group, preferably methyl. An advantage of the present embodiment is that the yield of the reaction is largely increased when compared to coupling spacers S where R₁ is H. This conclusion was not expected as in other metal catalyzed reactions (such as Suzuki coupling) this is not experimentally observed.

In accordance with a preferred embodiment of the present invention, at steps a) to d) n equals to 3 or 4. In case n equals to 3 tritopic linkers can be synthesized in accordance with the present invention. In case n equals 4, tetratopic linkers can be synthesized in accordance with the present invention. In accordance with the present embodiment MOFs having honeycomb morphology could be achieved in an easier way rather than from more commonly used linear ditopic linkers. Many linear organic linkers can grow MOF structures that interpenetrate each other. This can decrease the pore size, limiting the use of the material for some applications where a higher diffusion rate is needed. The MOF linkers synthesized in accordance with the present embodiment allow solving this problem. The shape of the linkers, together with the choice of metal cores, help growing MOFs in 2 dimensions, by fixing the length of one axis, preventing interpenetration. Using rigid (conjugated) organic linkers gives rise to geometrical constraints that favour the isoreticular principle.

In accordance with the present invention, the solvent utilized during the direct arylation reaction at step d) is an ether, preferably a cyclic ether. In an embodiment according of the present invention, at step d) the solvent is tetrahydrofuran (THF), also known as oxolane. It has been found that the use of ethers as solvents is preferred over e.g. other solvents such as DMF due to the fact that higher yields can be achieved and a more environmentally friendly solvent system can be used.

In accordance with the present invention, by means of the term “ether”, reference is made to a compound comprising a C—O—C bond. A cyclic ether is a compound wherein the C—O—C bond is part of a cyclic structure. Examples of ethers are tetrahydrofuran (THF), dioxane, diethylether, dimethylether, tetrahydropyrane, 2-methyl-tetrahydrofuran.

In a further embodiment according of the present invention, at step b) the second aryl moiety is substituted with a group C of formula (D). Therefore, in one embodiment each of the n spacers S comprises a group C which is connected to the second aryl group having a direct C—C bond with the first aryl group of the core. In other words, the molecular core X and the group C are directly connected to the same second aryl moiety. For example, when the spacer S is a single thiophene ring, the C group can and the B group can be directly connected to the thiophene aryl ring, at respectively positions 2 and 5, thereby being in accordance with the present embodiment.

In an embodiment according of the present invention, at step b) at least one spacer S, preferably all spacers S, comprise an amino group. The amino group can be mono- or di-substituted (—NHR or —NR₂) or unsubstituted (—NH₂), wherein each R can be alkyl and/or other groups. An advantage of this embodiment is that amino groups can function as tags allowing to assemble tagged MOFs which can be later on used to change the properties of the porous material in order to facilitate its use for certain applications. The use of —NH₂ tags according to the present embodiment has the advantage that no interference is caused with the process of MOF assembly.

In an embodiment according of the present invention, at step b) at least one spacer S, preferably all spacers S, comprise an amino group and/or a group C of formula (D) attached to the second aryl moiety. In accordance with the present embodiment, the second aryl moiety of the spacer S, which is the moiety which was directly connected to the core X via direct arylation, comprises amino group and/or a group C of formula (D). For example, in accordance with the present embodiment, the spacer S is methyl 3-(4-methylphenylsulfonamido)thiophene-2-carboxylate (6), which comprises a group B equals to H, an amino group (NH-Tos) and a methyl carboxylate group all attached to the same second aryl moiety, i.e. a thiophene ring. In this example, both the amino group and the group C of formula (D) are attached to the second aryl moiety.

In an embodiment according of the present invention, the second aryl moiety is a thiophene ring. It has been found that such second aryl moiety is advantageous, first, the thiophene ring has a strong preference for electrophilic aromatic substitution on the α positions of thiophene, compared with the β positions Kα/Kβ=100. This selectivity is significant because it will allow reaching higher yields in faster and easier pathways. Secondly, it is also possible to functionalize the β-positions of the thiophene ring selectively, in order to introduce a tag (e.g. —NH₂) in this position for the functionalization of MOFs derived therefrom.

In an embodiment according of the present invention, at steps a) to d), for each molecular core X, respectively spacer S, groups A and B are each independently selected from either H or Y, wherein Y is a direct arylation leaving group, such as a halide, such as Cl, Br, F, I, preferably Br, a triflate, a tosylate or a pseudohalide.

In a further embodiment according of the present invention, the second aryl moiety is a thiophene ring and B is H. Therefore, in accordance with the present embodiment, the thiophene ring is part of the spacer S and the group on the thiophene ring which is adapted to be removed during direct arylation is a hydrogen atom. It has been found and the present configuration allows for the direct arylation reaction to take place, due to CH activation on thiophene.

In an embodiment according of the present invention, at step d), the molar ratio of n first aryl moieties to spacers S is at least 1:1, preferably 1:2. It has been found that this molar ratio with an excess of spacers S, is beneficial in obtaining higher product yields. In other words, in accordance with the present embodiment, the direct arylation reaction is conducted so that for each first aryl moiety, each comprising a group A, at least the same number of second aryl moieties, each comprising a group B, is used. For example, in case a tritopic core is used, which comprises 3 first aryl moieties comprising e.g. 3 groups A equals to Br, in the direct arylation according to the present embodiment a number of second aryl moieties equals to at least 3 is provided. In case each spacer comprises a single aryl moiety comprising a group B, 3 spacers S are provided. E.g. Example 20 hereinafter is in accordance with the present embodiment, for which the molar ratio of n first aryl moieties to spacers S is 1:1, whereas Example 22 is in accordance with the preferred embodiment, for which the molar ratio of n first aryl moieties to spacers S is 1:2 (e.g. 3 first aryl moieties, 6 spacers). In accordance with a preferred embodiment of the invention, at step d), the molar ratio of n first aryl moieties to spacers S is at least equimolar (1:1), preferably spacers S are reacted in excess, preferably with a molar ratio 1:2

In a second aspect, the present invention pertains to a method of manufacturing of a MOF, the method comprising the steps a) to d) according to any one of the embodiments herein provided, and further comprising the steps of:
e) providing, if not already provided at step b), a free carboxylic group(s) (—COOH) from the group C according to formula (D) on any one, preferably all, of the spacers S;
f) coordinating a metal ion or cluster M with one or more of the free carboxylic groups provided at step e) or b), preferably all, of said spacers S, thereby forming the MOF.

In other words, in case R₁ equals to alkyl e.g. methyl, step e) comprises providing a carboxylic acid moiety e.g. by deprotecting the carboxylate, in case a free carboxylic acid moiety is not yet provided at step b). After the carboxylate moiety is freed, and therefore a free carboxylic group(s) (—COOH) is provided, the group C according to formula (D) is capable of coordinating a metal ion or cluster M, thereby forming the MOF.

In accordance with the present invention, by means of the term “metal ion or cluster M”, reference is made to a metal ion or metal cluster comprising La, Zr, In and Ga, such as La, In, and ZrO clusters.

In a third aspect, the present invention pertains to a linker obtained according to the method of any one of claims 1 to 10, comprising:
a molecular core X comprising n first aryl moieties, and
n spacers S, each comprising at least a second aryl moiety connected by means of direct C—C bond to any one of the first aryl moieties, and wherein each spacer S comprises a group C according to formula (D):

—COOR₁ (D)
wherein
R₁ is selected from the list: H, an alkyl group, preferably methyl;
n≥2.

In an embodiment according to the present invention, in the linker R₁ is an alkyl group, preferably methyl.

In an embodiment according to the present invention, in the linker n equals to 3 or 4.

### EXPERIMENTAL PART

### Materials and Methods

All chemical reagents and solvents, including tetrahydrofuran (THF) (99+%, stabilized with butylated hydroxytoluene—BHT) were purchased from commercial suppliers (Fluorochem, TCI Europe, VWR, Chem-Lab Analytical and Acros Organics) and used without further purification. 1H NMR spectra were recorded at 400 MHz with a Bruker Avance III HD 400 MHz spectrometer using deuterated chloroform (CDCl3) or dimethylsulfoxide-d6 (DMSO-d6) as the solvent and tetramethylsilane (TMS) as the internal standard. J values are quoted in Hertz. Column chromatography was performed on silica gel 60-200 mesh. TLC was performed on silica gel 60 F254. For PXRD, samples were transferred to a clay sample holder and measured between 1 and 80° 2θ on a Bruker D8 Eco equipped with Cu Kα radiation (λ=1.5406 Å). Porosity and surface area measurements were performed on a Quantachrome Quadrasorb SI (Quantachrome Instruments, Odelzhauzen, Germany) automated gas adsorption system using nitrogen as the absorbate at liquid nitrogen temperature (−196° C.). All the samples were outgassed in an AS-6 degasser under vacuum for 16 h at 120° C. before adsorption measurements. The surface area was calculated using the BET method in the range of relative pressure 0.015-1.

### Crystallography

Samples of the synthesized compounds were recrystallized by slow evaporation of a dichloromethane (DCM) solution, and suitable crystals mounted with grease on a borate glass capillary. Crystallographic data sets were measured at room temperature on a Rigaku R-Axis RAPID II image plate diffractometer, with a mirror-monochromated MM002+Cu Kα microsource, using ω scans. Images were integrated with the HKL3000 package v.707. Structure solution software was either SHELXS, SHELXT or SHELXD, structures were refined with SHELXL-2018/3 using the ShelxLE graphical interface. Static disorder, which occurs frequently in monosubstituted thiophene rings, was refined to the extent possible.

### Synthesis

### Example 1—1,3,5-Tri(4-bromophenyl)benzene (1)

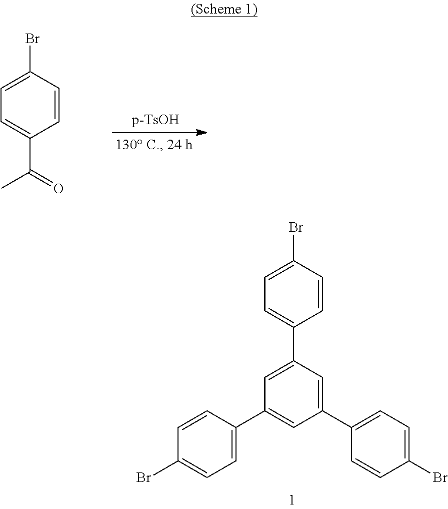

25 g of 4-bromo acetophenone and 2.5 g of p-toluene sulfonic acid monohydrate were put in a flask with a magnetic stirrer at 130° ° C. for 24 hours. When the reaction mixture had cooled down to room temperature, 20 mL of NaHCO₃ (5 g in 100 ml of water) were added to it. The precipitate was filtered and then washed once with water and once with ethanol (EtOH). Recrystallization from chloroform gave 13 g of orange needlelike crystals (yield 57%).

### Example 2—Methyl thiophene-2-carboxylate (2)

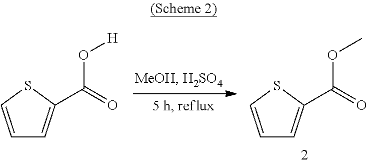

3.2 g of thiophene-2-carboxylic acid were dissolved in 25 mL of methanol. To this mixture 1.25 mL of H₂SO₄ were added. The reaction was run for 5 h under reflux conditions. After cooling down the reaction mixture was extracted with chloroform. The organic phase was washed with water, 5% NaHCO₃ (aqueous) and a saturated solution of NaCl. The solvent was evaporated under reduced pressure to give 3.3 g of the desired product as a yellow oil (92% yield).

### Example 3—2,2′-bithiophene-5-carboxylic acid (3)

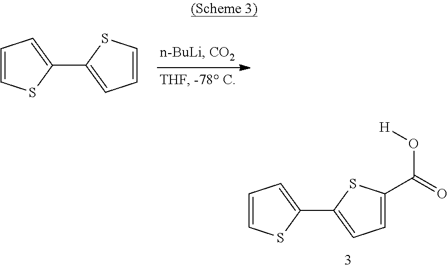 [SMILES: c1csc(-c2cccs2)c1 ; O=C(O)c1ccc(-c2cccs2)s1]

2.5 g of 2,2′-bithiophene was dissolved in 100 ml of dry THF (99+% stabilized with BHT) distilled from a mixture of Na and benzophenone, and placed in a 250 mL three neck flask. The mixture was kept stirring at −78° C. under nitrogen for 30 minutes. 8.2 mL of nbutyl-lithium (1.9M in hexanes) were added dropwise. The reaction was stirred for 30 minutes at −78° C. under nitrogen. An excess of solid CO₂ was added to the mixture and it was stirred for 30 more minutes at −78° C. The mixture was allowed to warm up to room temperature before the solvent was evaporated under reduced pressure, the resulting solid was dissolved in 1M NaOH. The remaining solid was filtered, and the filtrate acidified with HCl to obtain the product as a white precipitate that was filtered out of the solution, washed with water and dried on the air. 56 Yield 65%.

### Example 4—Methyl 2,2′-bithiophene-5-carboxylate (4)

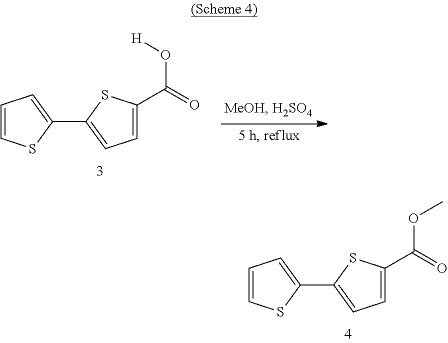

2.7 g of 2,2′-bithiophene-5-carboxylic acid (3) was dissolved in 130 ml of methanol and 6.4 mL of sulfuric acid. The mixture was stirred under reflux for 5 h. When the reaction was cooled down, the solvent was evaporated under reduced pressure. Water was added and the mixture was neutralized with sodium bicarbonate. Extraction with DCM was performed and the organic phases were washed with water, 5% sodium bicarbonate and saturated brine. The organic phases were dried over MgSO₄ and the solvent was evaporated. The crude was purified by column chromatography using DCM as eluent. Yield 91%.

### Example 5—Methyl 4-(2-thienyl)benzoate (5)

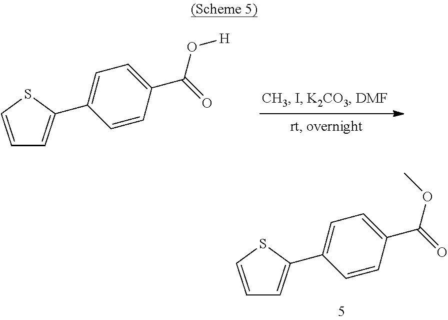

1 g of 4-(thiophen-2-yl)-benzoic acid was dissolved in 20 ml of dimethylformamide (DMF). 760 mg of K₂CO₃ and 760 mg of iodomethane were added. The reaction was stirred for 15 h at room temperature. The reaction mixture was poured in 10 ml of water, then it was extracted with chloroform. The organic phase was washed with water and a solution of saturated NaCl. The solvent was evaporated to give 1 g of product (94% yield).

### Example 6—Methyl 3-(4-methylphenylsulfonamido)thiophene-2-carboxylate (6)

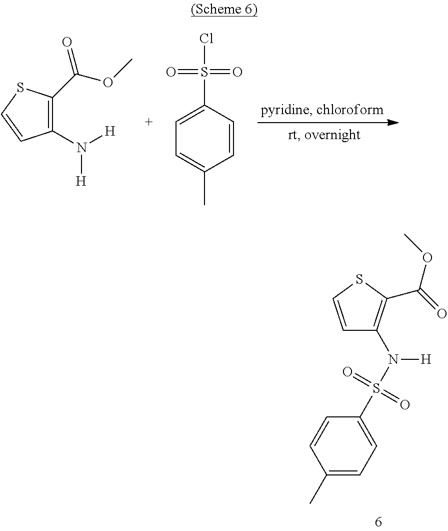

5 g of methyl 3-amino-2-thiophenecarboxylate were dissolved in 20 mL of chloroform. To this 6.67 g of 4-toluenesulfonyl chloride (1.1 eq.) and 3 g of pyridine (1.2 eq.) were added. The reaction was stirred at room temperature overnight. The reaction mixture was dried on the rotary evaporator, 15 mL of ethyl acetate was added and the reaction was washed with 4M HCl (15 mL×3). The organic phase was washed with NaHCO₃ (aq., saturated). The organic layer was dried over MgSO₄, filtered and the solvent was evaporated under vacuum. This gave 8.5 g of crystalline product (86% yield).

### Example 7—3-chloro-3-(2-thienyl)-2-propenenitrile (7)

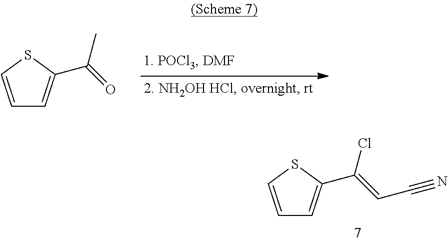

2 eq of POCl₃ (7.5 mL) were added dropwise to 13 mL of dry DMF (4 eq) keeping the temperature below 25° C. on an ice bath. This was stirred on the ice bath for 15 minutes. The reaction mixture was then heated up to 55° C. and 5 g (4.3 mL) of acetylthiopene (1 eq) was added dropwise keeping the temperature at 55° C. After stirring at 55° ° C. for 15 min the temperature was allowed to reach room temperature. The mixture was stirred for 30 minutes at room temperature. 30 more mL of DMF were added to the reaction mixture to be able to stir it. 11 g of NH₂OH·HCl (4 eq) was added portion-wise and the reaction was stirred at room temperature overnight. 150 ml of ice water was added and the precipitate was collected by vacuum filtration. The precipitate was washed three times with water. The product was purified by column chromatography using DCM as eluent. 670 mg of pure crystalline product was collected.

### Example 8—Methyl 4-amino[2,2′-bithiophene]-5-carboxylate (8)

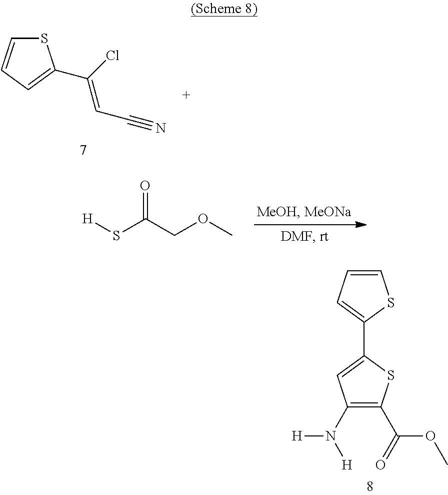

A solution of methylthioglycolate (1.3 eq, 0,545 mg, 2.2 ml) in 2.5 ml of methanol was added to a solution of sodium methoxide (0,005 mol, 0,277 g) in 2.5 ml of methanol at room temperature. This mixture was stirred for 1 h at room temperature. A solution of 3-chloro-3-(2-thienyl)-2-propenenitrile (7) (670 mg, 0.0039 mol) in DMF was added dropwise over 10 minutes at room temperature and then the reaction mixture was stirred at 60° C. for 2 h. The reaction mixture was allowed to reach room temperature. A solution of sodium methoxide in methanol was added dropwise at room temperature. This was stirred for 2 h at 60° ° C. The solution was poured in cold water and stirred for 10 minutes. Extraction with dichloromethane was performed and the DCM phase was washed with water, brine and dried over MgSO₄. Column chromatography using DCM was performed, to give 700 mg of product (with traces of methylthioglycolate).

### Example 9—Methyl 4-[[(4-methylphenyl)sulfonyl]amino][2,2′-bithiophene]-5-carboxylate (9)

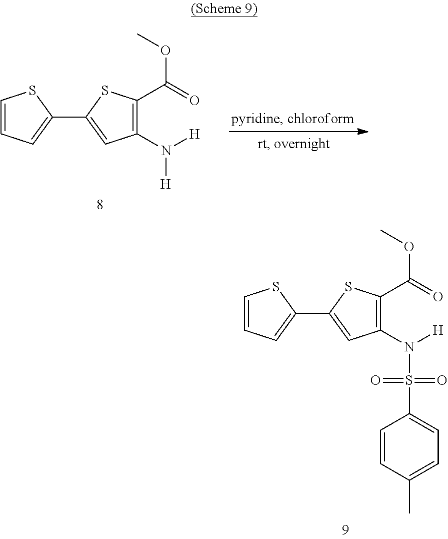 [SMILES: COC(=O)c1sc(-c2cccs2)cc1N ; COC(=O)c1sc(-c2cccs2)cc1NS(=O)(=O)c1ccc(C)cc1]

700 mg of methyl 4-amino[2,2′-bithiophene]-5-carboxylate (8) were dissolved in 10 ml of chloroform. To this 670 mg of 4-toluenesulfonyl chloride (1.2 eq.) and 280 UL of pyridine (1.2 eq.) were added. The reaction was stirred at room temperature overnight. The reaction mixture was concentrated on the rotary evaporator, ethyl acetate was added and the solution was washed with 4M HCl. The organic phase was separated and washed with NaHCO₃ (aq, saturated). The organic layer was dried with MgSO₄, filtered and concentrated on the rotary evaporator.

### Example 10—General procedure for the synthesis of star-shaped linkers—Methyl 5-[4-[3,5-bis[4-(5-methoxycarbonyl-2-thienyl)phenyl]phenyl]phenyl]thiophene-2-carboxylate (10)

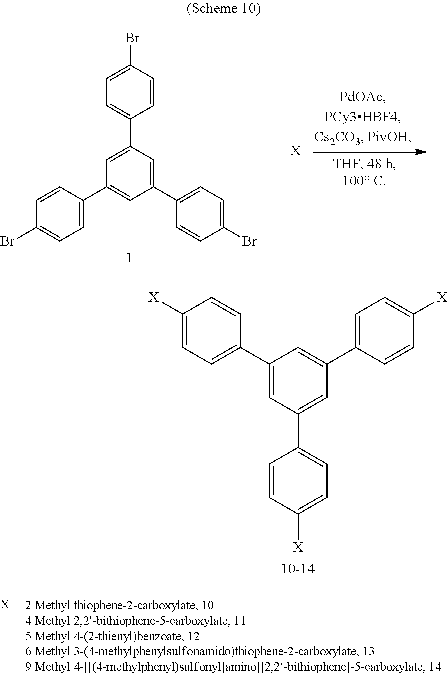

1 eq of 1,3,5-tri(4-bromophenyl)benzene (1) (250 mg), 6 eq of methyl thiophene-2-carboxylate (2) (386 mg), 3 eq. of Cs₂CO₃, 10 mol % Pd(OAc)₂, 20 mol % Pcy-HBF₄, 30 mol % pivalic acid (PivOH), 10 mL of THF. The reaction was stirred for 48 h at 100° ° C. in an autoclave. When the reaction was stopped, water was added to the reaction mixture. The reaction mixture was filtered and extracted with chloroform. The combined organic phases were washed with water, brine and dried over MgSO₄. The chloroform phase was evaporated under reduced pressure and the crude product was purified via column chromatography on silica gel using DCM as eluent. Yield 56%

### Example 11—Methyl 5-[5-[4-[3,5-bis[4-[5-(5-methoxycarbonyl-2-thienyl)-2-thienyl]phenyl]phenyl]phenyl]-2-thienyl]thiophene-2-carboxylate (11)

1 eq of 1 (250 mg), 6 eq of 4 (620 mg), 3 eq of Cs₂CO₃, 10 mol % Pd(OAc)₂, 20 mol % Pcy-HBF₄, 30 mol % PivOH, 10 mL of THF. Yield 41%

### Example 12 Methyl 4-[5-[4-[3,5-bis[4-[5-(4-methoxycarbonylphenyl)-2-thienyl]phenyl]phenyl]phenyl]-2-thienyl]benzoate (12)

1 eq of 1 (250 mg), 6 eq of 5 (603 mg), 3 eq of Cs₂CO₃, 10 mol % Pd(OAc)₂, 20 mol % Pcy-HBF₄, 30 mol % PivOH, 10 mL of THF. Yield 62%

### Example 13—Methyl 3-(benzenesulfonamido)-5-[4-[3,5-bis[4-[4-(benzenesulfonamido)-5-methoxycarbonyl-2-thienyl]phenyl]phenyl]phenyl]thiophene-2-carboxylate (13)

1 eq of 1 (250 mg), 6 eq of 6 (860 mg), 3 eq of Cs₂CO₃, 10 mol % Pd(OAc)₂, 20 mol % Pcy-HBF₄, 30 mol % PivOH, 10 mL of THF. Yield 64%

### Example 14—Methyl 5-[5-[4-[3,5-bis[4-[5-[5-methoxycarbonyl-4-(ptolylsulfonylamino)-2-thienyl]-2 thienyl]phenyl]phenyl]phenyl]-2-thienyl]-3-(ptolylsulfonylamino) thiophene-2-carboxylate (14)

1 eq of 1 (250 mg), 6 eq of 8 (1 g), 3 eq of Cs₂CO₃, 10 mol % Pd(OAc)₂, 20 mol % Pcy-HBF₄, 30 mol % PivOH, 10 mL of THF. Yield 15%

### Example 15—5-[4-[3,5-bis[4-(5-carboxy-2-thienyl)phenyl]phenyl]phenyl]thiophene-2-carboxylic acid (15)

1.2 g of 10 were dissolved in 400 ml of DMF. 100 mL of saturated NaOH solution was added. The reaction mixture was stirred overnight at room temperature. HCl was added to reach pH 1. The precipitate was filtered and washed with water to remove the salt. 1.18 g of pure product was recovered, yielding 97%.

### Example 16—5-[4-[3,5-bis[4-(5-carboxy-2-thienyl) phenyl]phenyl]phenyl]thiophene-2-carboxylic acid (15)

543 mg (1 eq.) 1,3,5-Tri(4-bromophenyl)benzene, 384 mg (3 eq.) Thiophene-2-carboxylic acid, (10 mol %) 70 mg of Pd(OAc)₂, 220 mg (20 mol %) of Pcy·HBF₄, 92 mg (30 mol %) of PivOH, 1,049 g (3 eq.) of Cs₂CO₃, 20 mL DMF, 3d, 100° C. The reaction was run under nitrogen. The reaction completion was followed by TLC. After three days the reaction was stopped and the reaction mixture was acidified with HCl to precipitate the product. 13 mg of solid (2% yield) were precipitated, dissolved in EtOH, then dried and sent to NMR. NMR showed a forest of peaks in both the aromatic and aliphatic regions, meaning that the solid was not the product and it was impure.

### Example 17—5-[4-[3,5-bis[4-(5-carboxy-2-thienyl)phenyl]phenyl]phenyl]thiophene-2-carboxylic acid (15)

543 mg (1 eq.) 1,3,5-Tri(4-bromophenyl)benzene, 384 mg (3 eq.) Thiophene-2-carboxylic acid, (10 mol %) 70 mg of Pd(OAc)₂, 220 mg (20 mol %) of Pcy·HBF₄, 92 mg (30 mol %) of PivOH, 1,049 g (3 eq.) of Cs₂CO₃, 20 mL DMF, 48 h, 100° C. The reaction was run under nitrogen. Water was added, the reaction mixture was filtered. The solid was washed with DMF and water. The water phase was extracted with chloroform. In the chloroform phase, DMF was left and when it was evaporated crystals of cesium carbonate were found. The water phase was acidified, the precipitate was filtered out from the mother solution and washed with water and ethanol. The mother solution and the water phase were evaporated, cesium carbonate was found in them. The EtOH wash fraction was evaporated, 36 mg were found and sent to NMR. From the NMR results, a peak at 9 ppm let us believe that DMF had reacted with carboxylic acid.

### Example 18 Methyl 5-[4-[3,5-bis[4-(5-methoxycarbonyl-2-thienyl)phenyl]phenyl]phenyl]thiophene-2-carboxylate (10)

530 (1 eq.) 1,3,5-Tri(4-bromophenyl)benzene, 515 (3 eq.) methyl thiophene-2-carboxylate, 63 mg (10 mol %) of Pd(OAc)₂, 209 mg (20 mol %) of Pcy·HBF₄, 86 mg (30 mol %) of PivOH, 1.02 g (3 eq.) of Cs₂CO₃, 20 mL DMF, 48 h, 100° C. Water was added to the reactions mixture. Extraction with chloroform was performed. The water phase was acidified, no precipitate was found. This led us to the conclusion that this time DMF had not reacted with the thiophene. The chloroform phase had starting materials (core and ester). The DMF was left in the chloroform and taken out to be evaporated separately, this gave 13 mg of material that was impure (checked on TLC). Because of the impurities, no NMR was measured for this sample.

### Example 19—5-[4-[3,5-bis[4-(5-carboxy-2-thienyl)phenyl]phenyl]phenyl]thiophene-2-carboxylic acid (15)

250 mg (1 eq.) 1,3,5-Tri(4-bromophenyl)benzene, 200 mg (3 eq.) Thiophene-2-carboxylic acid, 36 mg (10 mol %) of Pd(OAc)₂, 101 mg (20 mol %) of Pcy·HBF₄, 46 mg (30 mol %) of PivOH, 700 mg (3 eq.) of Cs₂CO₃, 20 mL THF, 48 h, 120° C. The reaction was run in a autoclave. Water was added to the reaction mixture, extraction with DCM was performed. The water phase was acidified, no precipitate was found. The water phase was then neutralized and evaporated, no organics were found. The DCM phase was evaporated, 5 mg of solid were found and measured with NMR. No product was found, the NMR spectrum showed hydrogens from the 1,3,5-Tri(4-bromophenyl)benzene and some peaks in the aliphatic region.

### Example 20 Methyl 5-[4-[3,5-bis[4-(5-methoxycarbonyl-2-thienyl)phenyl]phenyl]phenyl]thiophene-2-carboxylate (10)

250 mg (1 eq.) 1,3,5-Tri(4-bromophenyl)benzene, 210 mg (3 eq.) methyl thiophene-2-carboxylate, 37 mg (10 mol %) of Pd(OAc)₂, 102 mg (20 mol %) of Pcy·HBF₄, 42 mg (30 mol %) of PivOH, 500 mg (3 eq.) of Cs₂CO₃, 10 mL THF, 48 h, 100° C. The reaction was run in a autoclave. TLC showed that there was no starting material left. Water was added to the reaction mixture. Extraction with chloroform. Column chromatography with DCM as eluent was performed. 53 mg (16% yield) of product was obtained and confirmed by NMR.

### Example 21 Methyl 5-[4-[3,5-bis[4-(5-methoxycarbonyl-2-thienyl)phenyl]phenyl]phenyl]thiophene-2-carboxylate (10)

250 mg (1 eq.) 1,3,5-Tri(4-bromophenyl)benzene, 354 mg (6 eq.) methyl thiophene-2-carboxylate, 95 mg (30 mol %) of Pd(OAc)₂, 310 mg (60 mol %) of Pcy·HBF₄, 130 mg (90 mol %) of PivOH, 500 mg (3 eq.) of Cs₂CO₃, 10 mL THF, 48 h, 100° C. The reaction was run in a autoclave. (In this reaction, the amounts of catalyst, phosphine and pivalic acid were mistakenly 3 times more) Water was added to the reaction mixture. Extraction with chloroform was performed. The reaction mixture was purified via column chromatography using DCM as eluent. Fractions with pure product and product with some impurities were obtained. Yield 37% (112 mg) calculated only on pure fractions.

### Example 22 Methyl 5-[4-[3,5-bis[4-(5-methoxycarbonyl-2-thienyl)phenyl]phenyl]phenyl]thiophene-2-carboxylate (10)

1 eq of 1,3,5-tri(4-bromophenyl)benzene (265 mg), 6 eq of methyl thiophene-2-carboxylate (386 mg), 500 mg (3 eq.) of Cs₂CO₃, 32 mg (10 mol %) Pd(OAc)₂, 105 mg (20 mol %) Pcy-HBF₄, 44 mg (30 mol %) pivalic acid (PivOH), 10 mL of THF. The reaction was stirred for 48 h at 100° C. in an autoclave. When the reaction was stopped, water was added to the reaction mixture. The reaction mixture was filtered and extracted with chloroform. The combined organic phases were washed with water, brine and dried over MgSO₄. The chloroform phase was evaporated under reduced pressure and the crude product was purified via column chromatography on silica gel using DCM as eluent. 175 mg of pure product were collected. Yield 56%

### Example 23—5-[4-[3,5-bis[4-[5-carboxy-4-amino-2-thienyl]phenyl]phenyl]phenyl]-3-aminothiophene-2-carboxylic acid (16)

2.5 g of (13) were dissolved in 300 ml of DMF. 100 ml of saturated NaOH solution was added. The reaction mixture was stirred overnight at room temperature. HCl was added to reach pH 1. The precipitate was filtered and washed with water to remove the salt. The precipitate was filtered on silica gel using 1% of MeOH in DCM as eluent. The fraction that remained on the silica was recovered by washing with DMF. After solvent evaporation 360 mg of product was recovered, yielding 24% (calculated on the title compound). The deprotection of (13) gave rise to a mixture of three products according to NMR, still containing material with a partially tosylated amino group, and partially methylated carboxylic acid. It is possible to integrate the NMR peaks of NH-Tos, of the deprotected carboxylic acid (COOH), and of the methyl group of the protected carboxylic acid (COOMe) to indicate the relative ratios. They integrate respectively as 0.26 (NH-Tos, 1H) 1 (COOH, 1H) and 1.15 (COOMe, 3H), leading to a ratio of 0.26/1/0.35. In addition, this fraction which was flushed off the silica still contains a lot of free toluenesulfonic acid. Its methyl protons integrate, together with the methyl protons of the tosyl protecting group on the product, as 6.09 protons, 5.31 of which are of the methyl group of free toluenesulfonic acid (3H), leading to a ratio of 1.77:1 free toluene sulfonic acid: free linker carboxylic acid.

### Example 24—MOF Synthesis

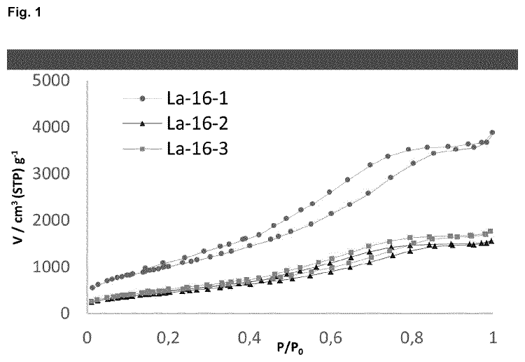

La-16-1. 50 mg of 16 (1 eq.) were stirred with 81 mg of Lanthanum(III) nitrate hydrate (La(NO₃)₃·xH₂O (5 eq.) in 2 mL of DMF for 30 minutes. La-16-2. 50 mg of 16 (1 eq.) were stirred with 81 mg of Lanthanum(III) nitrate hydrate (La(NO₃)₃·xH₂O (5 eq.) and 6 mg of benzoic acid (1 eq.) in 2 mL of DMF for 30 minutes. La-16-3. 50 mg of 16 (1 eq.) were stirred with 162 mg of Lanthanum(III) nitrate hydrate (La(NO₃)₃·xH₂O (10 eq.) and 60 mg of benzoic acid (10 eq.) in 2 mL of DMF for 30 minutes. The vials were transferred to an oven and warmed up to 85° C. at 2ºC/h. After 48 h the temperature was ramped down to room temperature at 4° C./h. The precipitates were centrifuged and washed three times with EtOH. The yields were 30, 2 and 30 mg for La-16-1, La-16-2 and La-16-3 respectively. N₂ sorption isotherms for La-16-1, La-16-2 and La-16-3 are shown in FIG. 1. The present example confirms that large pore MOFs can be synthesized starting from these star-shaped linkers. The sorption isotherms are of type IV, showing a large pore volume (5.67 cc/g for La-16-1) for the unmodulated synthesis, and a lower total pore volume (2.33 cc/g for La-16-2 and 2.62 cc/g for La-16-3) for the syntheses with benzoic acid present as a modulator, with surface areas of 3770, 1681 and 1831 m² g⁻¹ respectively The relatively gradual uptake suggests that the pore size distribution is not sharp. Pore size distributions are given in the SI.

### Example 25—Tris(4-bromophenyl)amine (TPB) (17)

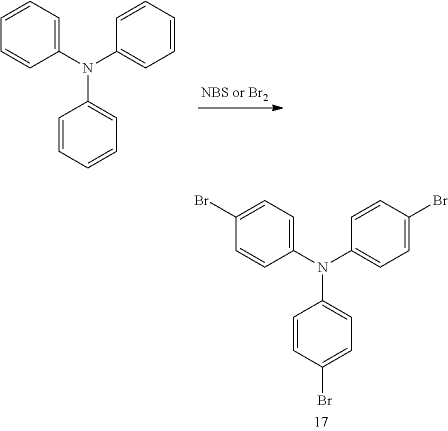 [SMILES: c1ccc(N(c2ccccc2)c2ccccc2)cc1 ; Brc1ccc(N(c2ccc(Br)cc2)c2ccc(Br)cc2)cc1]

Triphenylamine (1 g) were fully dissolved in a flask containing 6 ml CH₂Cl₂. Then Br₂ (0.6 ml) was dissolved in CH₂Cl₂ (1.6 ml) and the mixture was added drop wisely in above mentioned triphenylamine solution at 0° C. over a time period of 60 min. Then the mixture was kept and stirred for another 60 min at room temperature. After removing solvent the resulting solid was dissolved in a small amount of chloroform and of hot ethanol. After the solution is cooled in an ice bath, Tris(4-bromophenyl)amine crystallizes as colourless needles (94%).

### Example 26—2,4,6-tris(4-bromophenyl)-1,3,5-triazine (TBT)(18)

0.89 mL (0.01 mmol) trifluoromethanesulfonic acid was slowly added to 1 g (5.5 mmol) of 4-Bromobenzonitrile at 0° C. and stirred for 30 min. It was further stirred at room temperature overnight. The resulting mixture was washed with 100 ml of deionized water and filtered under vacuum. Purification of the product afforded of white solid in a yield of 98%.

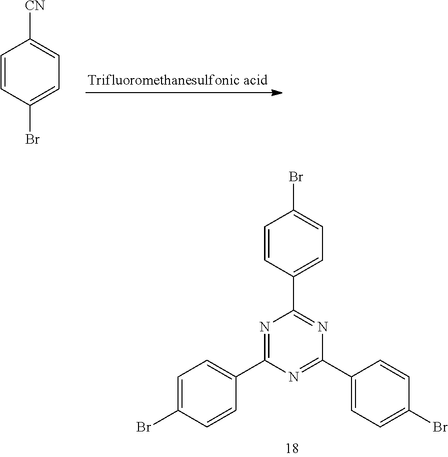 [SMILES: N#Cc1ccc(Br)cc1 ; Brc1ccc(-c2nc(-c3ccc(Br)cc3)nc(-c3ccc(Br)cc3)n2)cc1]

### Example 27—N,N,N′,N′-Tetrakis(4-bromophenyl)benzidine(DTPA)(20)

Tetraphenylbenzidine (TPA)(19): A solution of triphenylamine (1 g, 1 eq) in dry dichloromethane (50 mL) was prepared and cooled down to 0° C. Methanesulfonic acid (MSA) (5 mL, 19 eq) was added dropwise. After stirring for 2-3 minutes at 0° C. chloranil was added (2 g). The reaction mixture quickly turns deep blue. The reaction mixture is allowed to reach room temperature and the reaction completion is reached in about 5-10 minutes (TLC monitoring). The reaction is cooled down to 0° ° C. before being quenched by a saturated solution of NaHCO₃. The mixture is extracted with dichloromethane. The organic layers are gathered and washed with 2 M NaOH solution, washed with brine, dried over magnesium sulfate MgSO₄, filtered and evaporated under vacuum. (95% yield).

N,N,N′,N′-Tetrakis(4-bromophenyl)benzidine(DTPA)(20): To a 100 mL round-bottom flask were added 0.81 g, 1.66 mmol (TPA)(19) and 1.19 g, 6.66 mmol NBS. Chloroform (20 mL) was added, and the solution was stirred at room temperature (RT) for 1 h. And then 4.2 ml of acetic acid were added, and the solution was stirred for further 6.5 h at RT. The product was extracted with DCM, washed with water and brine twice and then the organic extract dried over anhydrous sodium sulfate. After solvent evaporation, the crude product was purified by column chromatography (20% DCM in heptane) to afford white solid (88%).

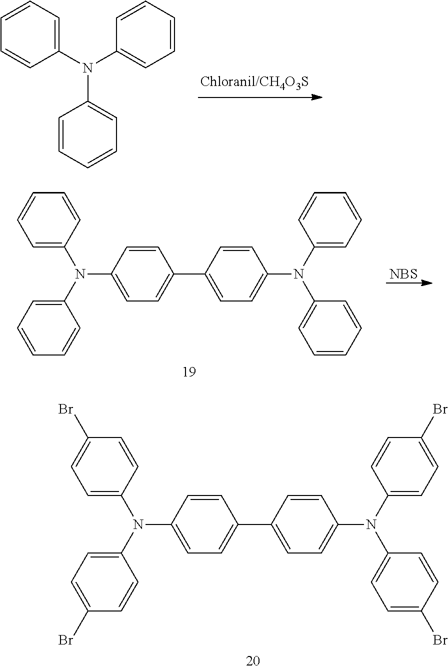

### Example 28—Tetrakis(4-bromophenyl) ethylene (21)

1 g of 4,4′ Dibromobenzophenone and 0.77 g of Zn powder was added to a 100 mL pre-dried three necked round-bottom flask with a magnetic stirrer. The flask was flushed with nitrogen for three times. 10 ml of dry THF was injected into the flask and the mixture was sealed under N₂ stream and stirred in an ice-bath for 0.5 h. Then 0.3 mL of TiCl₄ was injected drop-wise into the flask and the mixture was stirred again for 0.5 h on an ice bath. After 30 min stirring in ice bath the mixture was refluxed at 70° C. overnight. After cooling and solvent evaporation, 30 ml of deionized water and 6 mL of potassium carbonate solution were added to the reaction mixture. Then 6M HCl dropwise was added to the reaction mixture to adjust the pH of the solution to 7. The mixture was extracted with DCM and the organic layer was retained. Column chromatography was needed for purification (20% DCM in heptane) (Yield 70%).

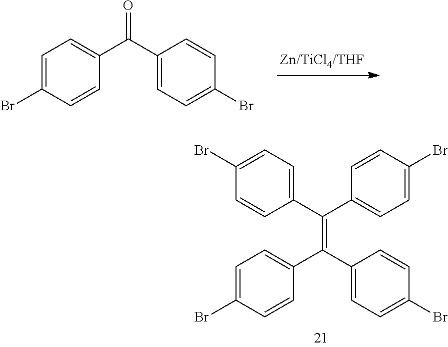 [SMILES: O=C(c1ccc(Br)cc1)c1ccc(Br)cc1 ; Brc1ccc(C(=C(c2ccc(Br)cc2)c2ccc(Br)cc2)c2ccc(Br)cc2)cc1]

### Example 29—Synthesis of MOF Linkers of 17, 18

1 eq of the tritopic core 17 or 18, 6 eq of a linear linker, 3 eq. of Cs₂CO₃, Pd(OAc)₂: 3×10% per reaction (respect to core), Pcy·HBF₄: 3×20% per reaction (respect to core), Pivalic acid: 3×30% per reaction (respect to core), THF. The reaction was stirred for 48 h at 100° C. in an autoclave. After cooling down the reaction mixture is washed with THF and water and then filtered. The filtrate is extracted with DCM. Column chromatography is used to purify the product by using DCM as a mobile phase

The following MOF linkers were obtained:

| Tritopic | | | |
|---|---|---|---|
| Core | | | |
| used | Linker used | MOF Linker obtained | Yield |
| 17 | 5- phenylthiophene- 2-carboxylic acid | 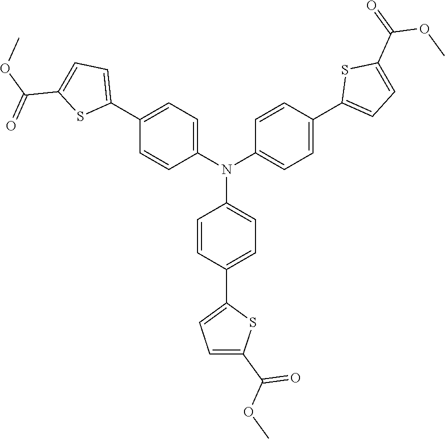 [SMILES: COC(=O)c1ccc(-c2ccc(N(c3ccc(-c4ccc(C(=O)OC)s4)cc3)c3ccc(-c4ccc(C(=O)OC)s4)cc3)cc2)s1] | 98% |
| 17 | Methyl 2,2′- bithiophene-5- carboxylate (4) | 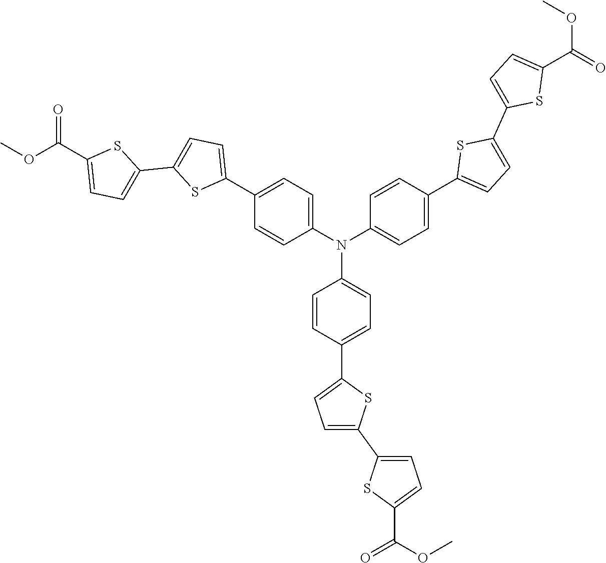 [SMILES: COC(=O)c1ccc(-c2ccc(-c3ccc(N(c4ccc(-c5ccc(-c6ccc(C(=O)OC)s6)s5)cc4)c4ccc(-c5ccc(-c6ccc(C(=O)OC)s6)s5)cc4)cc3)s2)s1] | 58% |
| 18 | Methyl thiophene- 2-carboxylate (2). | 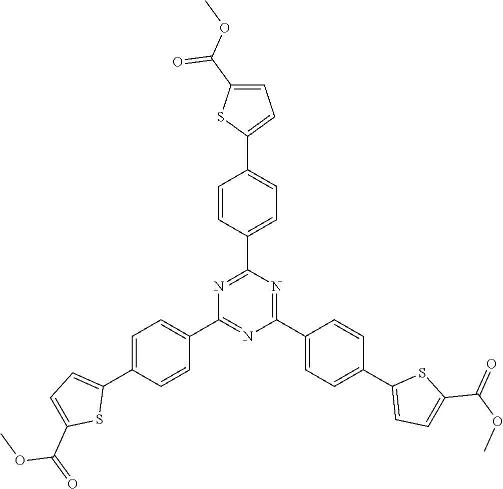 [SMILES: COC(=O)c1ccc(-c2ccc(-c3nc(-c4ccc(-c5ccc(C(=O)OC)s5)cc4)nc(-c4ccc(-c5ccc(C(=O)OC)s5)cc4)n3)cc2)s1] | 60% |

### Example 30—Synthesis of MOF Linkers of 20, 21

1 eq of the tritopic core 17 or 18, 8 eq of a linear linker, 4 eq. of Cs₂CO₅, Pd(OAc)₂: 4×10% per reaction (respect to core), Pcy·HBF₄: 4×20% per reaction (respect to core), Pivalic acid: 4×30% per reaction (respect to core), THF. The reaction was stirred for 48 h at 100° C. in an autoclave. After cooling down the reaction mixture is washed with THF and water and then filtered. The filtrate is extracted with DCM. Column chromatography is used to purify the product by using DCM as a mobile phase

The following MOF linkers were obtained:

| Tetratopic | | | |
|---|---|---|---|
| Core | | | |
| used | Linker used | MOF Linker obtained | Yield |
| 21 | Methyl thiophene-2- carboxylate (2). | 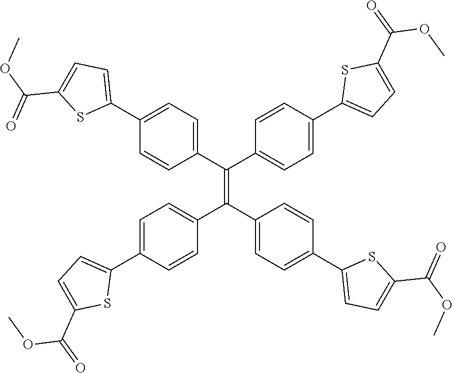 [SMILES: COC(=O)c1ccc(-c2ccc(C(=C(c3ccc(-c4ccc(C(=O)OC)s4)cc3)c3ccc(-c4ccc(C(=O)OC)s4)cc3)c3ccc(-c4ccc(C(=O)OC)s4)cc3)cc2)s1] | 51% |
| 20 | Methyl thiophene-2- carboxylate (2). | 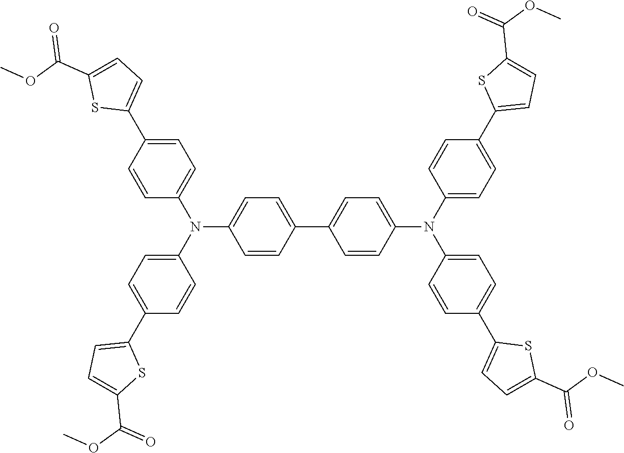 | 55% |
| 21 | 5- phenylthiophene- 2-carboxylic acid | 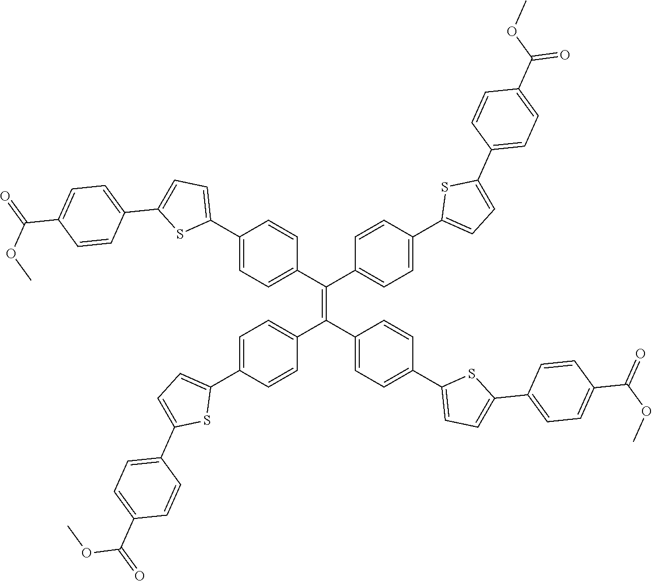 [SMILES: COC(=O)c1ccc(-c2ccc(-c3ccc(C(=C(c4ccc(-c5ccc(-c6ccc(C(=O)OC)cc6)s5)cc4)c4ccc(-c5ccc(-c6ccc(C(=O)OC)cc6)s5)cc4)c4ccc(-c5ccc(-c6ccc(C(=O)OC)cc6)s5)cc4)cc3)s2)cc1] | 41% |

### REFERENCES

1. Kajiwara, Takashi & Higashimura, Hideyuki & Higuchi, Masakazu & Kitagawa, Susumu. (2017). Design and Synthesis of Porous Coordination Polymers with Expanded One-Dimensional Channels and Strongly Lewis-Acidic Sites. ChemNanoMat. 4. 10.1002/cnma.201700256.

1. A method of manufacturing of a linker, comprising the steps of:
a) providing a molecular core X comprising n first aryl moieties having a group A attached thereto, wherein n≥2,
b) providing at least n spacers S, each comprising at least a second aryl moiety having a group B attached thereto, and wherein each of said n spacers S is substituted with a group C according to formula (D): 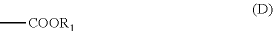
wherein
R₁ is selected from the list: H, an alkyl group, preferably methyl;
c) catalytically activating group A of the molecular core X and group B of the spacers S by means of a cross coupling metal catalyst, preferably comprising a metal selected from the list: Palladium, Copper, Nickel, Iron, Ruthenium, Rhodium, Cobalt, or combinations thereof, preferably Palladium; and
d) reacting via direct arylation in a solvent the molecular core X comprising n first aryl moieties with n second aryl moieties part of the spacers S, thereby forming at least a C—C bond at the position of said group A of the molecular core X and group B of the spacers S, and wherein the solvent is an ether, preferably a cyclic ether, and wherein
at steps a) to d), for each molecular core X, respectively spacer S, groups A and B are each selected from either H or Y, wherein at least one is H, and wherein Y is a direct arylation leaving group, selected from the list comprising: a halide, a triflate, a tosylate, a pseudohalide.

2. The method according to any one of the previous claims, wherein at step d) the solvent is tetrahydrofuran (THF).

3. The method according to any one of the previous claims, wherein at step b) the second aryl moiety is substituted with a group C of formula (D).

4. The method according to any one of the previous claims, wherein at step b) at least one spacer S, preferably all spacers S, comprise an amino group.

5. The method according to any one of the previous claims, wherein at step b) at least one spacer S, preferably all spacers S, comprise an amino group and/or a group C of formula (D) attached to the second aryl moiety.

6. The method according to any one of the previous claims, wherein the second aryl moiety is a thiophene ring.

7. The method according to claim 6, wherein the second aryl moiety is a thiophene ring and B is H.

8. The method according to any one of the previous claims, wherein at step d), the molar ratio of n first aryl moieties to spacers S is at least 1:1, preferably 1:2.

9. A method of manufacturing of a MOF, the method comprising the steps a) to d) according to any one of claims 1 to 8, and further comprising the steps of:
e) providing, if not already provided at step b), a free carboxylic group(s) (—COOH) from the group C according to formula (D) on any one, preferably all, of the spacers S;
f) coordinating a metal ion or cluster (M) with one or more of the free carboxylic groups provided at step e) or b), preferably all, of said spacers S, thereby forming the MOF.

10. A linker obtainable according to the method of any one of claims 1 to 9, comprising:
a molecular core X comprising n first aryl moieties, and
n spacers S, each comprising at least a second aryl moiety connected by means of direct C—C bond to any one of the first aryl moieties, and wherein each spacer S comprises a group C according to formula (D): 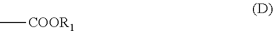
wherein
R₁ is selected from the list: H, an alkyl group, preferably methyl;
n≥2.

11. The linker according to claim 10, wherein at least one spacer S, preferably all spacers S, comprise an amino group, preferably attached to the second aryl moiety.

12. The linker according to any one of claims 10 to 11, wherein the second aryl moiety is selected from: thiophene, 2,2′-Bithiophene, substituted or unsubstituted.

13. The linker according to any one of claims 10 to 12, wherein the molecular core X is selected from the list comprising: 1,1,2,2-Tetraphenylethylene, Pyrene, para-Terphenyl, Triphenylamine, 2,4,6-Triphenyl-1,3,5-triazine, N,N,N′,N′-Tetraphenylbenzidine, substituted or unsubstituted.

14. The linker according to claim 13, wherein the molecular core X is selected from: Triphenylamine, 2,4,6-Triphenyl-1,3,5-triazine, N,N,N′,N′-Tetraphenylbenzidine, substituted or unsubstituted.

15. The linker according to claim 10, having formula: 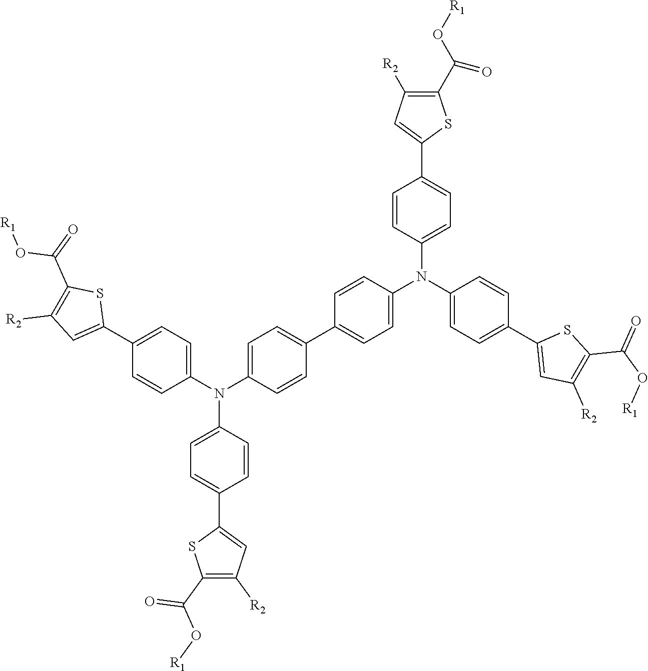
wherein R₁ is selected from the list: H, an alkyl group, preferably methyl.

16. The linker according to claim 10 of formula: 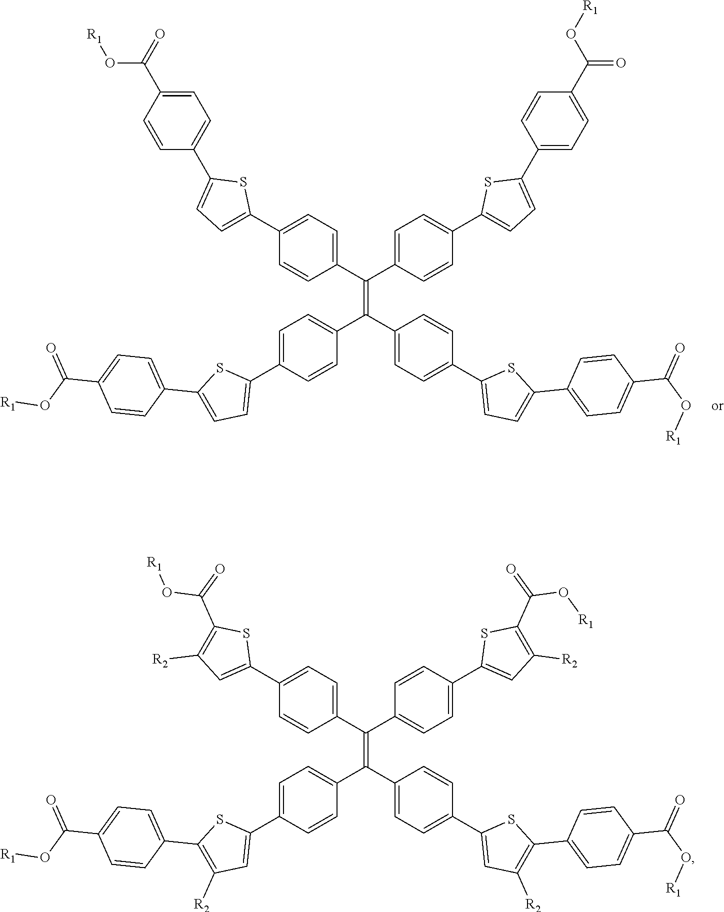
wherein
R₁ is selected from the list: H, an alkyl group, preferably methyl;
R₂ is selected from the list: H, amino group.

17. The linker according to claim 10 of formula: 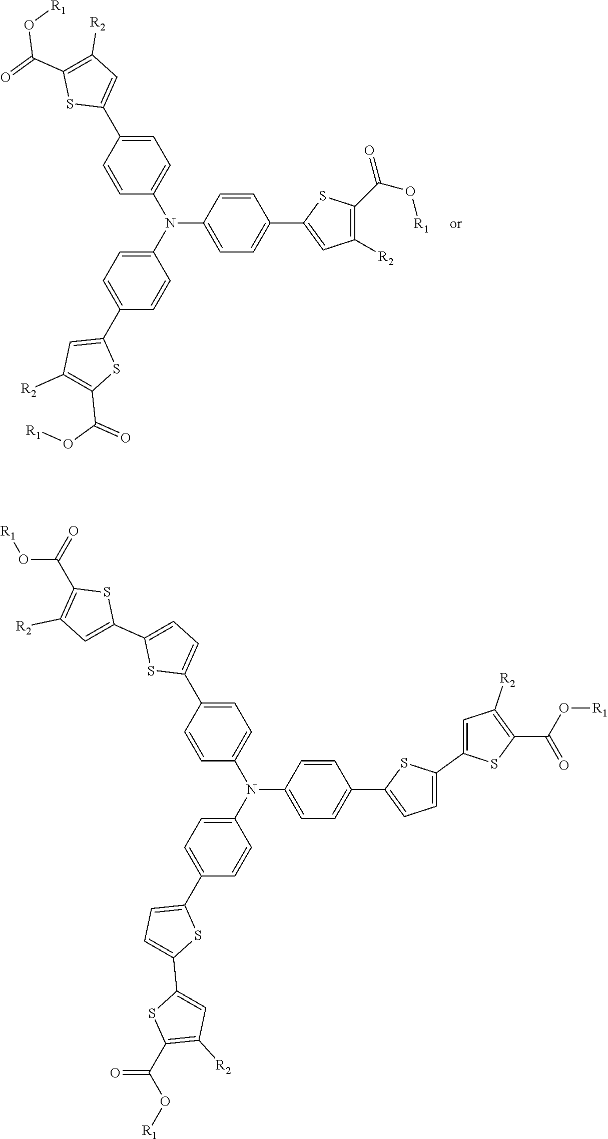
wherein
R₁ is selected from the list: H, an alkyl group, preferably methyl;
R₂ is selected from the list: H, amino group.

18. The linker according to claim 10 of formula: 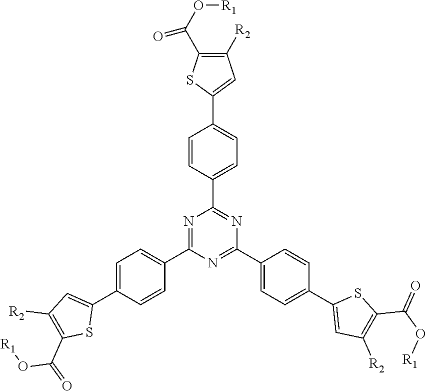
wherein
R₁ is selected from the list: H, an alkyl group, preferably methyl;
R₂ is selected from the list: H, amino group.

19. The linker according to claim 10 of formula: 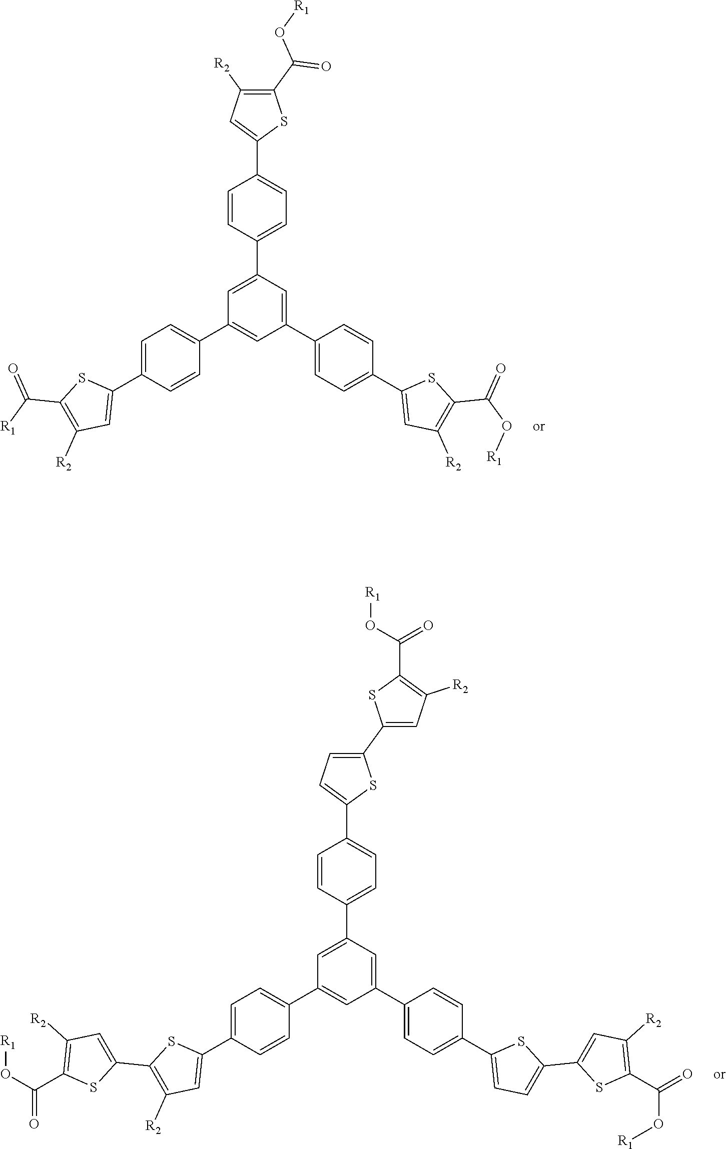 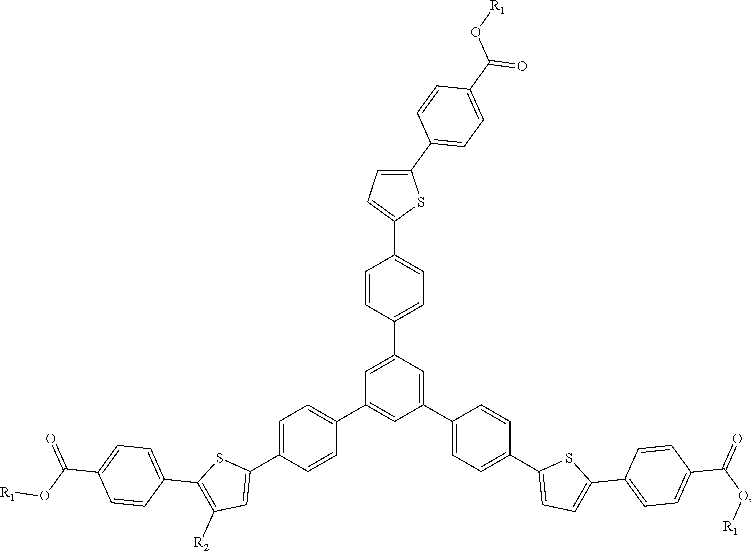
wherein
R₁ is selected from the list: H, an alkyl group, preferably methyl;
R₂ is selected from the list: H, amino group.
